(19) United States
(12) Patent Application Publication
(10) Pub. No.: US 2021/0337786 A1
(43) Pub. Date: Nov. 4, 2021

(54) COMPOSITION FOR DECONTAMINATING SOLID SURFACES

(71) Applicant: SOCIETE D'EXPLOITATION DE PRODUITS POUR LES INDUSTRIES CHIMIQUES SEPPIC, Paris Cedex 07 (FR)

(72) Inventor: Marie-Francoise CHIRAC, Paris (FR)

(21) Appl. No.: 17/284,356

(22) PCT Filed: Oct. 9, 2019
(86) PCT No.: PCT/FR2019/052391

§ 371 Date: Apr. 9, 2021

(30) Foreign Application Priority Data
Oct. 12, 2018 (FR) 1859465

(51) Int. Cl. A01N 25/16; A01N 59/00; A01N 25/24; A01P 1/00; A61L 2/22

(52) U.S. Cl. CPC A01N 25/16; A01N 59/00; A61L 2101/06; A01P 1/00; A61L 2/22; A01N 25/24

(57) ABSTRACT

Disclosed is a composition for decontaminating solid surfaces. The composition includes a surfactant mixture based on cocoyl glutamate and alkyl polyglycosides, at least one thickening agent and/or gelling agent, and at least one decontamination agent.

### CROSS-REFERENCE TO RELATED APPLICATIONS

This application is the U.S. national phase of International Application No. PCT/FR2019/052391 filed Oct. 9, 2019 which designated the U.S. and claims priority to FR 1859465 filed Oct. 12, 2018, the entire contents of each of which are hereby incorporated by reference.

### BACKGROUND OF THE INVENTION

### Field of the Invention

The subject of the present invention is a composition, a foam comprising at least one decontamination agent, and a process for decontaminating solid surfaces.

The composition according to the invention makes it possible to obtain an aqueous foam, comprising decontamination agents, which remains stable and which generates a height or a volume of foam in a rapid time.

The present invention is of application in the decontamination of solid surfaces contaminated for example by grease, by radioactive mineral deposits. When the decontamination agents are disinfectants, the foams according to the invention comprising them are used for the disinfection of solid surfaces contaminated by pathogenic agents such as bacteria, fungi and viruses.

### Description of the Related Art

Numerous foaming compositions and foams already exist which are intended for the treatment of solid surfaces, and more particularly for treatments for cleaning and treatments for disinfecting solid surfaces.

However, these foams have the drawback of rapidly destabilizing by draining water. Indeed, foams consist of a set of gaseous cells separated by thin layers of liquids, formed by the juxtaposition of bubbles generated by a gas dispersed in a liquid. They are generally prepared from aqueous compositions comprising at least one foaming surfactant by mixing a gas, for instance air or nitrogen or carbon dioxide. Certain surfactants are known to generate foams by mixing with gases.

In operations for cleaning and disinfecting solid surfaces, the foam constitutes the carrier of the aqueous solution comprising the decontaminating agent and its stability characteristic must be such as to induce a sufficient contact time with the solid surface to allow a effective decontamination treatment.

Thus, when these foams are used for cleaning and/or disinfecting solid surfaces that are easy to access, such as for example solid surfaces of external equipment which may comprise horizontal walls, it is necessary for the foam to show high stability so that the treatment undertaken is effective. Likewise, when these foams are used for cleaning and/or disinfecting solid surfaces consisting of vertical walls, or else of internal walls of equipment of a vertical nature, or else of walls internal to these complex tubular pipes with convolutions such as elbows or coils, it is essential for said foams that are used to be highly stable.

Indeed, if the foam used is not sufficiently effective due to its instability, it is necessary:
either to apply this foam repeatedly to the solid surface to be treated, and consequently to generate a larger amount of effluents,
or to increase the concentration of decontaminating agent in the foam, which generates an additional cost and, depending on the types of solid surfaces, which may generate unwanted corrosion phenomena.

However, the mechanical properties of these foams are different depending on the nature of said surfactants. Thus, to obtain foams of great stability, which are characterized by a high foam viscosity and a high duration, those skilled in the art must combine the surfactant with one or more additives having the effect of increasing the rigidity of the structure of the gaseous cells.

The international application published under number WO 93/22538 A1 describes a tunnel excavation process characterized by the injection of a foam generated by activation of a foaming surfactant in aqueous solution in the presence of a foaming aid chosen from the group consisting of fatty amines, fatty alcohols, fatty alkanol amides or tertiary amine oxides. However, these foam-stabilizing additives are often poorly biodegradable and are sometimes toxic, which makes them noncompliant with the new environmental regulatory provisions.

The international patent application published under number WO 2008/101855A1 discloses decontamination foams having properties improved over those previously described.

International application WO 2008/101855A1 provides stabilized foams for the treatment of solid surfaces comprising decontamination agents and solid stabilizing agents; said stabilized foams no longer necessarily comprising surfactants.

The solid stabilizers of such foams are thermoplastic and/or thermosetting polymers or copolymers, such as, for example, polyolefins, polyacrylics or polyurethanes. However, given the changes in the regulations regarding the dissemination of plastics in nature, cosmetic industries are looking to use ingredients that are non-plastic in nature.

The international patent application published under number WO 2018/115635A1 discloses mixtures (M₁) comprising surfactants based on N-acylated derivatives of glutamic acid and/or aspartic acid, in acid form and/or or in salified form, and surfactants of the alkylpolyglycoside type.

The international patent application published under number WO 2018/115635A1 more particularly describes solutions comprising mixtures (M₁) as described above, compositions comprising water, mixtures (M₁), at least one fluorinated surfactant and at least one gelling agent, and the use of such solutions and such compositions for preparing foams intended for fighting fires. Such surfactant mixtures (M₁) generate foams which are particularly suitable for fighting fires because they are obtained after a short period of expansion, and they have a stability suitable for preventing restart of the fire.

However, in the presence of a decontaminating agent, the mixtures (M₁), their solutions and their thickened solutions, as described in international patent application WO 2018/115635A1, generate a volume of foam which is reduced relative to the volume of foam generated in the absence of such decontaminating agents.

There is therefore a real need to have a thickened foaming composition which makes it possible to overcome the drawbacks of the thickened foaming compositions of the prior art, that is to say which makes it possible in particular to prepare decontaminating gelled foams with a short expansion time, to extend the life of said decontaminating gelled foam, to use such compositions for cleaning and/or disinfecting solid surfaces contaminated by dirt and/or by pathogenic agents according to processes which make it possible to reduce the amount of effluents, to use less corrosive decontamination agents, to use these agents at a lower concentration, and also to reduce the difficulty, the pollution and also the cost of the treatment.

### SUMMARY OF THE INVENTION

A solution of the present invention is a composition (C_{D}) for decontaminating solid surfaces, comprising, per 100% of its mass:

a)—from 35% to 99.3% by mass, preferably from 37% to 98.7% by mass and even more preferentially from 40% to 97.6% by mass of water;
b)—from 0.5% to 40% by mass, preferably from 1% to 40% by mass, and even more preferentially from 2% to 40% by mass of at least one decontamination agent;
c)—from 0.1% to 10% by mass, preferably from 0.1% to 8% by mass and even more particularly from 0.2% to 8% by mass of at least one gelling agent and/or thickener (AG);
d)—from 0.1% to 15% by mass, preferably from 0.2% to 15% by mass, and even more preferentially from 0.2% to 12% by mass of a mixture (M₁) comprising, per 100% of its own mass:
(i)—from 50% to 99% by mass, preferably from 65% to 85% by mass and even more preferentially of a composition (C₁) comprising, per 100% of its mass:

(α)—from 65% to 90% by mass, preferably from 65% to 85% by mass and even more preferentially from 65% to 80% of at least one compound of formula (I):

R₁—C(═O)—NH—CH(COOH)—(CH₂)₂—COOH (I)

in acid or partially or totally salified form, wherein the group R₁—C(═O)— represents a linear or branched, saturated or unsaturated acyl radical including from 8 to 18 carbon atoms, and

(β)—from 10% to 35% by mass, preferably from 15% to 35% by mass and even more preferentially from 20% to 35% by mass of at least one compound of formula (II):

R₁—C(═O)—OH (II)

in acid or partially or totally salified form, wherein the group R₁ is as defined for formula (I),
(ii)—from 1% to 50% by mass, preferably from 1% to 45% by mass and even more preferentially from 1% to 43% by mass of a composition (C₂) comprising, per 100% of its mass:

(γ)—from 14% to 80% by mass of a composition (C₃) or of a mixture of compositions (C₃), said composition (C₃) being represented by formula (III):

R₃—O-(G₃)ₚ-H (III)

wherein R₃ represents a linear or branched, saturated or unsaturated aliphatic radical including from 12 to 16 carbon atoms, G₃ represents the residue of a reducing sugar and p represents a decimal number greater than or equal to 1.05 and less than or equal to 5;

(δ)—from 20% to 80% of a composition (C₄) or of a mixture of compositions (C₄), said composition (C₄) being represented by formula (V):

R₄—O-(G₄)_{q}-H (V)

wherein R₄ represents a linear aliphatic radical, chosen from n-butyl (n-C₄H₉—), n-pentyl (n-C₅H₁₁—), n-hexyl (n-C₆H₁₃—) and n-heptyl (n-C₇H₁₅—) radicals, G₄ represents a reducing sugar residue and q represents a decimal number greater than or equal to 1.05 and less than or equal to 5.

It should be noted that the composition according to the invention will preferably be in foaming form.

In the context of the present invention, the term “compounds of formula (I) or (II) in acid or partially or totally salified form” means that one, several or all of the carboxyl functions present in one or other of said compounds of formula (I) or (II) is either in acid form (—COOH) or in salified form (—COO⁻M⁺). In the latter case, M⁺ represents a monovalent cation chosen from:
the ammonium cation,
monovalent cations of alkali metals, for example the sodium (Na⁺), potassium (K⁺) or lithium (Li⁺) cation,
(hydroxyalkyl)ammonium, bis(hydroxyalkyl)ammonium or tris(hydroxyalkyl)ammonium cations wherein the hydroxyalkyl radical(s) include from 1 to 4 carbon atoms, for example 2-hydroxyethanammonium, 2-hydroxypropanammonium, bis(2-hydroxyethyl)ammonium and tris(2-hydroxyethyl)ammonium cations,
(alkyloxyalkyl)ammonium, bis(alkyloxyalkyl)ammonium or tris(alkyloxyalkyl)ammonium cations wherein the alkyloxyalkyl radical(s) include from 2 to 6 carbon atoms, for example the 2-ethoxyethanammonium cation,
(hydroxyalkylaminoalkyl)ammonium, bis(hydroxyalkylaminoalkyl)ammonium or tris(hydroxyalkylaminoalkyl)ammonium cations wherein the hydroxyalkylaminoalkyl radical(s) include from 2 to 6 carbon atoms, for example the 2-hydroxyethylaminomethanammonium cation and the 2-hydroxyethylaminoethanammonium cation.

Depending on the case, the composition according to the invention may have one or more of the characteristics below:
the decontamination agent is chosen from the elements of the group consisting of acidic decontamination agents, in acidic or partially or totally salified form; basic decontamination agents; disinfectants;
the gelling agent and/or thickener is chosen from polysaccharides consisting of monosaccharide derivatives, polysaccharides consisting solely of monosaccharides, cellulose and cellulose derivatives, starches and linear or branched or crosslinked polyelectrolytes;
said composition (C₃) consists of a mixture of compounds represented by formulae (III₁), (III₂), (III₃), (III₄) and (III₅):

R₃—O-(G₃)₁-H (III₁),

R₃—O-(G₃)₂-H (III₂),

R₃—O-(G₃)₃-H (III₃),

R₃—O-(G₃)₄-H (III₄),

R₃—O-(G₃)₅-H (III₅),

in the respective molar proportions a₁, a₂, a₃, a₄ and as such that:
the sum a₁+a₂+a₃+a₄+a₅ is equal to 1, and
the sum a₁+2a₂+3a₃+4a₄+5a₅ is equal to p;
said composition (C₄) consists of a mixture of compounds represented by formulae (V₁), (V₂), (V₃), (V₄) and (V₅):

R₄—O-(G₄)₁-H (V₁),

R₄—O-(G₄)₂-H (V₂),

R₄—O-(G₄)₃-H (V₃),

R₄—O-(G₄)₄-H (V₄),

R₄—O-(G₄)₅-H (V₅),

in the respective molar proportions a′₁, a′₂, a′₃, a′₄ and a′₅, such that:
the sum a′₁+a′₂+a′₃+a′₄+a′₅ is equal to 1, and
the sum a′₁+2a′₂+3a′₃+4a′₄+5a′₅ is equal to q;
the composition (C₂) comprises from 0% to 3% by mass of at least one alcohol of formula (IV):

R₃—OH (IV)

wherein R₃ is as defined in formula (III), and/or from 0% to 3% by mass of at least one alcohol of formula (VI):

R₄—OH (VI)

wherein R₄ is as defined for formula (V);
the compounds of formula (I) and of formula (II) are partially or totally salified in sodium salt or potassium salt form;
said mixture (M₁) comprises, per 100% of its mass, from 55% to 99% by mass of said composition (C₁) and from 1% by mass to 45% by mass of said composition (C₂), and more particularly from 57% to 99% by mass of said composition (C₁) and from 1% by mass to 43% by mass of said composition (C₂);
the composition (C₁) as defined previously comprises, per 100% of its mass, from 65% to 90% by mass of one or more compounds of formula (I) and from 10% to 35% by mass of one or more compounds of formula (II); and more particularly from 65% to 85% by mass of one or more compounds of formula (I) and from 15% to 35% by mass of one or more compounds of formula (II);
in formulae (I) and (II), the group R₁—C(═O)— represents an acyl radical chosen from octanoyl, decanoyl, dodecanoyl, tetradecanoyl, hexadecanoyl, octadecanoyl, 9-octadecenoyl, 9,12-octadecadienoyl and 9,12,15-octadecatrienoyl radicals;
the composition (C₁) included in the surfactant mixture (M₁), the compound(s) of formula (I) are chosen from monosodium N-cocoyl glutamate, monopotassium N-cocoyl glutamate, disodium N-cocoyl glutamate and dipotassium N-cocoyl glutamate;
said composition (C₂) comprises, per 100% of its mass:
   (γ)—a mass proportion of said composition (C₃) of greater than or equal to 14% and less than 70%, and
   (δ)—a mass proportion of said alcohol of formula (IV) of greater than or equal to 0% and less than or equal to 3%,
   (ε)—a mass proportion of said composition (C₄) of greater than or equal to 30% and less than or equal to 80%, and
   (η)—a mass proportion of said alcohol of formula (VI) of greater than or equal to 0% and less than or equal to 3%;
said composition (C₂) comprises, per 100% of its mass, a proportion by mass of said composition (C₃) greater than or equal to 14% and less than or equal to 65%, a proportion by mass of said alcohol of formula (IV) greater than or equal to 0% and less than or equal to 3%, a proportion by mass of said composition (C₄) greater than or equal to 35% and less than or equal to 80% and a proportion by mass of said alcohol of formula (VI) greater than or equal to 0% and less than or equal to 3%;
G₃ represents the residue of a reducing sugar chosen from glucose, xylose and arabinose residues;
p represents a decimal number greater than or equal to 1.05 and less than or equal to 2.5, more particularly greater than or equal to 1.05 and less than or equal to 2.0 and even more particularly greater than or equal to 1.25 and less than or equal to 2.0;
in formulae (III) and (IV) R₃ represents a linear alkyl radical chosen from n-dodecyl (n-C₁₂H₂₅—), n-tetradecyl (n-C₁₄H₂₉—) and n-hexadecyl (n-C₁₆H₃₂—) radicals;
G₄ represents the residue of a reducing sugar chosen from glucose, xylose and arabinose residues;
q represents a decimal number greater than or equal to 1.05 and less than or equal to 2.5, more particularly greater than or equal to 1.05 and less than or equal to 2.0 and even more particularly greater than or equal to 1.25 and less than or equal to 2.0;
in formulae (V) and (VI), R₄ represents a linear alkyl radical chosen from the n-hexyl (n-C₆H₁₃) and n-heptyl (n-C₇H₁₅) radicals; preferably, in formulae (V) and (VI), R₄ represents the heptyl (n-C₇H₁₅—) radical; according to another preferential embodiment, in formulae (V) and (VI), R₄ represents the n-hexyl (n-C₆H₁₃—) radical;
said composition (C₂) comprises a mixture of compositions (C₃) and compositions (C₄), said mixture comprising, per 100% of its mass:
   (γ₁)—from 13.6% to 44.4% by mass of a composition (C₃) represented by formula (III) wherein R₃ represents the (n-C₁₂H₂₅—) radical,
   (γ₂)—from 5% to 16.25% by mass of a composition (C₃) represented by formula (III) wherein R₃ represents the n-tetradecyl (n-C₁₄H₂₉) radical, and
   (γ₃)—from 1.4% to 4.55% by mass of a composition (C₃) represented by formula (III) wherein R₃ represents the n-hexadecyl (n-C₁₆H₃₂) radical,
   (ε₁)—from 35% to 80% by mass of a composition (C₄) represented by formula (V) wherein R₄ represents the n-heptyl radical (n-C₇H₁₅) radical;
said composition (C₂) comprises a mixture of compositions (C₃) and compositions (C₄), said mixture comprising, per 100% of its mass:
   (γ₁)—from 13.6% to 44.4% by mass, more particularly from 17% to 44.4% by mass, of a composition (C₃) represented by formula (III) wherein R₃ represents the n-dodecyl (n-C₁₂H₂₅) radical,
   (γ₂)—from 5% to 16.25% by mass, more particularly from 6.25% to 16.25% by mass, of a composition (C₃) represented by formula (III) wherein R₃ represents the n-tetradecyl (n-C₁₄H₂₉) radical, and
   (γ₃)—from 1.4% to 4.55% by mass, more particularly from 1.75% to 4.55% by mass, of a composition (C₃) represented by formula (III) wherein R₃ represents the n-hexadecyl (n-C₁₆H₃₂) radical, and
   (ε₁)—from 35% to 80% by mass, more particularly from 35% to 75% by mass, of a composition (C₄) represented by formula (V) wherein R₄ represents the n-hexyl (n-C₆H₁₃) radical;
in formula (III), G₃ represents a reducing sugar residue chosen from glucose, xylose and arabinose residues, p represents a decimal number greater than or equal to 1.05 and less than or equal to 2.5, more particularly greater than or equal to 1.05 and less than or equal to 2.0 and even more particularly greater than or equal to 1.25 and less than or equal to 2.0, and R₃ represents a linear alkyl radical chosen from n-dodecyl (n-C₁₂H₂₅), n-tetradecyl (n-C₁₄H₂₉—) and n-hexadecyl (n-C₁₆H₃₂) radicals;
in formula (III), G₃ represents a glucose residue, p represents a decimal number greater than or equal to 1.05 and less than or equal to 2.5, and R₃ represents a linear alkyl radical chosen from n-dodecyl (n-C₁₂H₂₅), n-tetradecyl (n-C₁₄H₂₉) and n-hexadecyl (n-C₁₆H₃₂) radicals;
in formula (III), G₃ represents a xylose residue, p represents a decimal number greater than or equal to 1.05 and less than or equal to 2.5, and R₃ represents a linear alkyl radical chosen from n-dodecyl (n-C₁₂H₂₅) radicals, the n-tetradecyl (n-C₁₄H₂₉) radical and the n-hexadecyl (n-C₁₆H₃₂) radical;
in formula (V), G₄ represents a reducing sugar residue chosen from glucose, xylose and arabinose residues, q represents a decimal number greater than or equal to 1.05 and less than or equal to 2.5, more particularly greater than or equal to 1.05 and less than or equal to 2.0 and even more particularly greater than or equal to 1.25 and less than or equal to 2.0, and R₄ represents an aliphatic alkyl radical chosen from n-hexyl (n-C₆H₁₃) radicals and the n-heptyl (n-C₇H₁₅—) radical;
in formula (V), G₄ represents a glucose residue, q represents a decimal number greater than or equal to 1.05 and less than or equal to 2.5, and R₄ represents an aliphatic alkyl radical chosen from n-hexyl (n-C₆H₁₃) and n-heptyl (n-C₇H₁₅) radicals;
in formula (V), G₄ represents a glucose residue, q represents a decimal number greater than or equal to 1.05 and less than or equal to 2.5, and R₄ represents the n-heptyl (n-C₇H₁₅) radical;
in formula (V), G₄ represents a xylose residue, q represents a decimal number greater than or equal to 1.05 and less than or equal to 2.5, and R₄ represents an aliphatic alkyl radical chosen from n-hexyl (n-C₆H₁₃) and n-heptyl (n-C₇H₁₅) radicals;
in formula (V), G₄ represents a xylose residue, q represents a decimal number greater than or equal to 1.05 and less than or equal to 2.5, and R₄ represents the n-heptyl (n-C₇H₁₅) radical;
the mass ratio:
   Δ=Mass of compound(s) of formula (I)/[Mass of composition (C₃)+Mass of composition (C₄)], is greater than or equal to 20/80 and less than or equal to 65/35, more particularly greater than or equal to 25/75 and less than or equal to 65/35;
   the mass ratio:
   Δ₁=Mass of composition (C₃)/Mass of composition (C₄), is greater than or equal to 20/80 and less than or equal to 70/30, more particularly greater than or equal to 20/80 and less than or equal to 65/35, even more particularly greater than or equal to 25/75 and less than or equal to 65/35;
at least one gelling agent and/or thickener (AG) chosen from xanthan gum (G_{X}), acacia gum exudate (G_{A}), the mixture of xanthan gum (G_{X}) and of acacia gum exudate (G_{A}) in a mass ratio between the xanthan gum (G_{X}) and the acacia gum exudate (G_{A}) which is greater than or equal to 1/3 and less than or equal to 3/1;
at least decontamination agent is a disinfectant chosen from the elements of the group consisting of chlorinated products, aldehydes and oxidizing agents;
at least decontamination agent is an acidic agent in acid or partially or totally salified form, chosen from the elements of the group consisting of hydrochloric acid, nitric acid, sulfuric acid, phosphoric acid, oxalic acid, benzoic acid, sorbic acid, dehydroacetic acid and peracetic acid;
at least decontamination agent is a basic agent chosen from the elements of the group consisting of sodium hydroxide, potassium hydroxide, sodium carbonate and potassium carbonate;
the composition comprises per 100% of its mass:
   a)—from 35% to 99.3% of water;
   b)—from 0.5% to 40% by mass of at least one decontamination agent which is a disinfectant chosen from the group consisting of hydrogen peroxide and sodium hypochlorite;
   c)—from 0.1% to 10% by mass of at least one gelling agent and/or thickener (AG) chosen from the elements of the group consisting of xanthan gum (G_{X}), acacia gum exudate (G_{A}), the mixture of xanthan gum (G_{X}) and of acacia gum exudate (G_{A}) in a mass ratio between the xanthan gum (G_{X}) and the acacia gum exudate (G_{A}) which is greater than or equal to 1/3 and less than or equal to 3/1;
   d)—from 0.1% to 15% by mass of said mixture (M₁) wherein:
the compound of formula (I) is chosen from monosodium N-cocoyl glutamate, monopotassium N-cocoyl glutamate, disodium N-cocoyl glutamate and dipotassium N-cocoyl glutamate,
the compound of formula (II) is chosen from sodium cocoate and potassium cocoate,
in formula (III), R₃ represents a linear or branched, saturated or unsaturated aliphatic radical including from 12 to 16 carbon atoms, G₃ represents a glucose or xylose residue and p represents a decimal number greater than or equal to 1.05 and less than or equal to 2.5;
in formula (IV), R₃ represents a linear or branched, saturated or unsaturated aliphatic radical including from 12 to 16 carbon atoms,
in formula (V), R₄ represents the n-heptyl (n-C₇H₁₅) radical, G₄ represents a glucose or xylose residue and q represents a decimal number greater than or equal to 1.05 and less than or equal to 2,
in formula (VI), R₄ represents the n-heptyl (n-C₇H₁₅) radical;
the composition comprises per 100% of its mass:
   a)—from 35% to 99.3% of water;
   b)—from 0.5% to 40% by mass of at least one decontamination agent which is an acidic agent in acid or partially or totally salified form, chosen from the group consisting of benzoic acid, sorbic acid and dehydroacetic acid;
   c)—from 0.1% to 10% by mass of at least one gelling agent and/or thickener (AG) chosen from the elements of the group consisting of xanthan gum (G_{X}), acacia gum exudate (G_{A}), the mixture of xanthan gum (G_{X}) and of acacia gum exudate (G_{A}) in a mass ratio between the xanthan gum (G_{X}) and the acacia gum exudate (G_{A}) which is greater than or equal to 1/3 and less than or equal to 3/1;
   d)—from 0.1% to 15% by mass of said mixture (M₁) wherein:
the compound of formula (I) is chosen from monosodium N-cocoyl glutamate, monopotassium N-cocoyl glutamate, disodium N-cocoyl glutamate and dipotassium N-cocoyl glutamate,
the compound of formula (II) is chosen from sodium cocoate and potassium cocoate,
in formula (III), R₃ represents a linear or branched, saturated or unsaturated aliphatic radical including from 12 to 16 carbon atoms, G₃ represents a glucose or xylose residue and p represents a decimal number greater than or equal to 1.05 and less than or equal to 2.5;
in formula (IV), R₃ represents a linear or branched, saturated or unsaturated aliphatic radical including from 12 to 16 carbon atoms,
in formula (V), R₄ represents the n-heptyl (n-C₇H₁₅) radical, G₄ represents a glucose or xylose residue and q represents a decimal number greater than or equal to 1.05 and less than or equal to 2,
in formula (VI), R₄ represents the n-heptyl (n-C₇H₁₅) radical;

The term “reducing sugar residue” denotes, in the definition of residues (G₃) and (G₄) of formulae (III) and (V) as defined previously, residues of saccharide derivatives without a glycoside bond established between an anomeric carbon and the oxygen of an acetal group, as defined in the reference publication: “Biochemistry, Daniel Voet/Judith G. Voet, page 250, John Wiley & Sons, 1990.”

The oligomeric structures (G₃)ₚ and (G₄)_{q} may be in any isomeric form, whether it is optical isomerism, geometrical isomerism or regioisomerism; it may also represent a mixture of isomers.

In formula (III) as defined above, the group R₃ is linked to G₃ via the anomeric carbon of the saccharide residue, so as to form an acetal function. Similarly, in formula (V) as defined above, the group R₄ is linked to G₄ via the anomeric carbon of the saccharide residue, so as to form an acetal function.

In formula (III) of the mixture (M₁) as defined above, G₃ and G₄, which are identical or different, may optionally represent, independently of one another, a reducing sugar residue, glucose, dextrose, sucrose, fructose, idose, gulose, galactose, maltose, isomaltose, maltotriose, lactose, cellobiose, mannose, ribose, xylose, arabinose, lyxose, allose, altrose, dextran and tallose.

The term “linear or branched, saturated or unsaturated aliphatic radical including from 12 to 16 carbon atoms” notably denotes for R₃ in formulae (III) and (IV):
a linear alkyl radical chosen from n-dodecyl (n-C₁₂H₂₅—), n-tetradecyl (n-C₁₄H₂₉—) and n-hexadecyl (n-C₁₆H₃₂—) radicals,
a branched alkyl radical derived from the isoalkanols of formula (1):

(CH₃)(CH₃)CH—(CH₂)ᵣ—CH₂—OH (1)

wherein r represents an integer between 8 and 16, for example the isododecyl, isotridecyl, isotetradecyl, isopentadecyl or isohexadecyl radical;
a branched alkyl radical derived from a Guerbet alcohol of formula (2):

CH(CₛH₂ₛ₊₁)(CₜH₂ₜ₊₁)—CH₂—OH (2)

wherein t is an integer between 2 and 12, s is an integer between 2 and 14 and the sum s+t is greater than or equal to 10 and less than or equal to 14, for example the 2-ethyldecyl, 2-butyloctyl, 2-ethyldodecyl, 2-butyldecyl, 2-hexyloctyl, 2-hexadecyl or 2-butyldodecyl radical.

The compounds of formula (I) as described previously are generally obtained by N-acylation of the corresponding amino acids or of salts thereof. It is described, for example, in the international patent application published under the number WO 98/09611. It is performed equivalently on an amino acid or on an amino acid mixture. The acylating agent generally consists of an activated derivative of the carboxylic acid of formula:

R₁—C(═O)—OH,

wherein R₁ is as defined previously, such as a symmetrical anhydride of this acid, the methyl ester of this acid, or an acid halide such as the acid chloride or the acid bromide. It may also consist of a mixture of activated derivatives of carboxylic acids obtained from natural oils or fats of animal or plant origin such as coconut kernel oil, coconut oil, palm kernel oil, palm oil, soybean oil, rapeseed oil, corn oil, beef tallow, spermaceti oil or herring oil.

It should be noted that composition (C₁) may be obtained by a process comprising at least:
one step A) of acylation of a compound of formula (VII):

NH₂—CH(COOH)—(CH₂)₂—COOH (VII),

in acid or partially or totally salified form, with a mixture of acid chlorides comprising, per 100 mol %, from 40 mol % to 60 mol % of dodecanoyl chloride, from 10 mol % to 20 mol % of tetradecanoyl chloride, from 5 mol % to 15 mol % of decanoyl chloride and from 5 mol % to 15 mol % of octanoyl chloride, and optionally and up to a maximum of 100 mol % of hexadecanoyl chloride and/or of octadecanoyl chloride and/or of 9-octadecenoyl chloride and/or of octadeca-9,12-dienoyl chloride.

Preferably, the mixture of acid chlorides used comprises, per 100 mol %, 11 mol % of octanoyl chloride, 9.5 mol % of decanoyl chloride, 51 mol % of dodecanoyl chloride, 15.5 mol % of tetradecanoyl chloride, 6.5 mol % of hexadecanoyl chloride, 2 mol % of octadecanoyl chloride, 3 mol % of 9-octadecenoyl chloride and 1.5 mol % of octadeca-9,12-dienoyl chloride.

It should also be noted that the composition (C₃) may be obtained by implementing a process comprising at least one step A′) of glycosylation of:
one to five molar equivalents, more particularly from two to four molar equivalents, even more particularly from three to four molar equivalents, of a mixture of alcohols comprising, per 100 mol %:
   from 40 mol % to 90 mol % of 1-dodecanol, more particularly from 65 mol % to 90 mol % and even more particularly from 75 mol % to 90 mol %,
   from 9 mol % to 40 mol % of 1-tetradecanol, more particularly from 9 mol % to 20 mol % and even more particularly from 9 mol % to 20 mol %, and
   from 1 mol % to 20 mol % of 1-hexadecanol, more particularly from 1 mol % to 15 mol % and even more particularly from 1 mol % to 5 mol %,
   with one molar equivalent of a reducing sugar of formula (VII):

HO-(G₃)-H (VII)

wherein G₃ represents a reducing sugar residue chosen from glucose, xylose and arabinose residues.

Likewise, it should also be noted that the composition (C₄) may be obtained by implementing a process comprising at least one step A₁′ of glycosylation of:
one to four molar equivalents, more particularly from one to three molar equivalents, even more particularly from two to three molar equivalents of at least one alcohol of formula (VI) with 1 molar equivalent of a reducing sugar of formula (VIII):

HO-(G₄)-H (VIII)

wherein G₄ represents a reducing sugar residue chosen from glucose, xylose and arabinose residues.

According to another particular aspect, a subject of the invention is said composition (C_{D}) as defined above, characterized in that the composition (C₃) is obtained by performing a process comprising at least one step A′ of glycosylation generally performed with mechanical stirring, by bringing 1 molar equivalent of a reducing sugar (G₃) into contact with from 1 to 5 molar equivalents of the mixture of 1-dodecanol, 1-tetradecanol and 1-hexadecanol as described previously, in the presence of an acidic catalytic system, under predetermined temperature and partial vacuum conditions.

Similarly, step A₁′ of the process for preparing composition (C₄), as defined previously, is generally performed with mechanical stirring, by bringing 1 molar equivalent of a reducing sugar (G₄) into contact with from 1 to 4 molar equivalents of at least one alcohol of formula (V), in the presence of an acidic catalytic system, under predetermined temperature and partial vacuum conditions.

Such temperature and partial vacuum conditions are, for example, temperature values of between 70° C. and 130° C. and a partial vacuum of between 300 mbar (3×10⁴ Pa) and 20 mbar (2×10³ Pa). The implementation of step A′ and of step A₁′ of glycosylation makes it possible to form, respectively, composition (C₃), i.e. a mixture of compounds represented by the formulae (III₁), (III₂), (III₃), (III₄) and (III₅) as defined previously, and optionally of an excess of the alcohol of formula (IV) or of the mixture of alcohols of formula (IV), and composition (C₄), i.e. a mixture of compounds represented by the formulae (V₁), (V₂), (V₃), (V₄) and (V₅) as defined previously, and optionally of an excess of the alcohol of formula (VI).

If necessary or if desired, step A′ or step A₁′ of the process for preparing the respective compositions (C₃) and (C₄) as defined previously may be followed, respectively, by a step B′ or a step B₁′ of removal of the alcohols, respectively, of formula (IV), or of the mixture of alcohols of formula (IV), and of formula (VI), which have not reacted during step A′) or step A₁′.

Such a preparation process may be completed, if necessary or if desired, by neutralization, filtration and decolorization operations.

The term “acidic catalytic system” denotes, in step A′ and in step A₁′ of the process defined above, strong acids such as sulfuric acid, hydrochloric acid, phosphoric acid, nitric acid, methanesulfonic acid, para-toluenesulfonic acid, trifluoromethanesulfonic acid, hypophosphorous acid, hyponitrous acid, polyphosphoric acid, or ion-exchange resins.

During step B′) or step B′1) of the process as described above, the alcohols, respectively, of formula (IV), or the mixture of alcohols of formula (IV) or the alcohol of formula (V) are removed according to methods known to those skilled in the art, for instance by distillation, such as thin-film distillation, molecular distillation or solvent extraction.

In the context of the present invention, the term “thickener” present in the composition (C_{D}) that is a subject of the present invention denotes a chemical compound or a chemical composition which increases the viscosity of the medium into which it is introduced.

In the context of the present invention, the term “gelling agent” present in the composition (C_{D}) that is a subject of the present invention denotes a chemical compound or a chemical composition which transforms a liquid medium into a structured state, which does not flow, by formation of a three-dimensional network within the liquid; the gel being considered as an intermediate state between the liquid state and the solid state.

In the context of the present invention, the term “polysaccharides” denotes saccharide polymers. The IUPAC definition of saccharides designates monosaccharides, compounds of monosaccharides per se and derivatives thereof, obtained either by reduction of a carbonyl group, or by oxidation of one or more hydroxyl functions, or by the replacement of one or more hydroxyl functions with a hydrogen atom, an amine group, a phosphate function, or a sulfate function. The polysaccharides most commonly used for preparing industrial, food, cosmetic or pharmaceutical compositions predominantly consist of monosaccharides, such as glucose, galactose, mannose or of monosaccharide derivatives for which the hydroxyl function of the terminal carbon has been oxidized to a carboxyl function. Two distinct groups may be distinguished among the polysaccharides: polysaccharides consisting solely of monosaccharides (or poly-monosaccharides) and polysaccharides consisting of monosaccharide derivatives.

According to a particular aspect, the gelling agents and/or thickeners present in the composition (C_{D}) that is a subject of the present invention are chosen from polysaccharides consisting solely of monosaccharides (or poly-monosaccharides).

Among the polysaccharides composed solely of monosaccharides, a distinction may be made between glucans, which are homopolymers of glucose which are very abundant in nature, glucomannoglycans, xyloglycans and galactomannans, which are polymers, the main chain of which consists of D-mannose units, joined together at β-1,4, and on which D-galactose units are grafted laterally by α-1,6 bonds. Galactomannans are present in several plant species, and more particularly in the leguminous species in which they constitute the albumen of the seeds. Depending on their plant origin, the degree of substitution (DS) of the D-galactose units on the D-mannose main chain of galactomannans ranges between 0 and 1:
galactomannans originating from cassia gum have a degree of substitution (DS) of approximately 1/5, meaning the lateral grafting of one D-galactose unit every 5 D-mannose units present on the main chain of the polysaccharide;
galactomannans originating from locust bean gum have a degree of substitution (DS) of approximately 1/4, meaning the lateral grafting of one D-galactose unit every 4 D-mannose units present on the main chain of the polysaccharide;
galactomannans originating from tara gum have a degree of substitution (DS) of approximately 1/3, meaning the lateral grafting of one D-galactose unit every 3 D-mannose units present on the main chain of the polysaccharide;
galactomannans originating from guar gum have a degree of substitution (DS) of approximately 1/2, meaning the lateral grafting of one D-galactose unit every 2 D-mannose units present on the main chain of the polysaccharide;
galactomannans originating from fenugreek gum have a degree of substitution (DS) of approximately 1/1, meaning the lateral grafting of one D-galactose unit for practically every D-mannose unit present on the main chain of the polysaccharide.

According to a more particular aspect, the gelling agents and/or thickeners present in the composition (C_{D}) that is a subject of the present invention are chosen from polysaccharides consisting solely of monosaccharides (or poly-monosaccharides) included in the group consisting of galactomannan originating from tara gum, galactomannan originating from guar gum and galactomannan originating from locust bean gum.

According to another particular aspect, the gelling agents and/or thickeners present in the composition (C_{D}) that is a subject of the present invention are chosen from polysaccharides consisting of monosaccharide derivatives. Among the polysaccharides consisting of monosaccharide derivatives, a distinction may be made between:
sulfated galactans, which are polymers of galactose which may have pendant sulfate-ester groups, represented notably by algal polysaccharides such as carrageenans and agar;
uronans, which are the polymers of uronic acids such as algins and pectins;
heteropolymers of monosaccharides and uronic acids: often of complex composition, these polymers are found notably in sap exudates (for instance gum arabic exudate and karaya gum exudate), but they are also produced by microorganisms, for instance xanthan gum and gellan gum;
glucosaminoglycans which are polysaccharides formed from a glucose derived by replacing its C-2 hydroxyl with an amine (referred to as 2-amino-2-deoxy-D-glucose or, more simply, glucosamine). The amine function may also be acetylated. Among the hydrocolloids in this class are chitosan, formed solely of glucosamine units, and hyaluronan, the repeating unit of which is a dimer of glucosamine and glucuronic acid.

Xanthan gum (G_{X}) has in recent decades become the microbial polysaccharide that is the most widely used in industry. Xanthan is a polysaccharide synthesized by bacteria of the genus Xanthomonas and, commercially, only the species X. campestris is used. The main chain of (G_{X}) is identical to that of cellulose, i.e. it is formed from β-D-glucose units connected together via carbons 1 and 4. There is one branched triholoside every two glucose units in the main chain, in a regular alternating manner; each branch consisting of a triholoside composed of two mannoses and a glucuronic acid, of the type: β-D-Manp-(1→4)-β-D-GlcAp-(1→2)-α-D-Manp-(1→3) [I. Capron et al., “About the native and renaturated conformation of xanthan exopolysaccharide”. 1997]. Xanthan gum (GX) is available in the form of a sodium, potassium or calcium salt.

Acacia gum is a complex, branched polysaccharide, the main chain of which consists of β-D-galactose units connected together via carbons 1 and 3. The chains branched to the main chain consist of β-D-galactose units connected together by carbons 1 and 6, also bearing α-arabinose units, and to a lesser extent β-glucoronosyl units. Both the main chain and the pendant chains contain α-L-arabinosyl, α-L-rhamnopyranosyl, β-D-glucuronopyranosyl and 4-O-methyl-β-D-glucuronopyranosyl units.

According to a more particular aspect, the gelling agents and/or thickeners present in the composition (C_{D}) that is a subject of the present invention are polysaccharides consisting of monosaccharide derivatives chosen from the elements of the group consisting of carrageenans, agar, algins, pectins, gum arabic exudate, karaya gum exudate, xanthan gum, gellan gum, chitosan and hyaluronan, and/or mixtures thereof.

According to an even more particular aspect, the gelling agents and/or thickeners present in the composition (C_{D}) that is a subject of the present invention are polysaccharides consisting of monosaccharide derivatives chosen from the elements of the group consisting of acacia gum exudate, karaya gum exudate and xanthan gum, and/or mixtures thereof.

According to an even more particular aspect, the gelling agents and/or thickeners present in the composition (C_{D}) that is a subject of the present invention are polysaccharides consisting of monosaccharide derivatives chosen from the elements of the group consisting of acacia gum exudate (G_{A}), xanthan gum (G_{X}), the mixture of xanthan gum (G_{X}) and acacia gum exudate (G_{A}) used in a mass ratio between the xanthan gum (G_{X}) and the acacia gum exudate (G_{A}) of greater than or equal to 1/3 and less than or equal to 3/1, sold notably by the company SEPPIC under the brand name Solagum™ AX.

According to a particular aspect, the gelling agents and/or thickeners present in the composition (C_{D}) that is a subject of the present invention are chosen from cellulose and cellulose derivatives.

In the context of the present invention, the term “cellulose” denotes a polysaccharide consisting of a linear chain of D-glucose molecules, the average molecular mass of which is at least 10 000 g·mol⁻¹, more particularly at least 15 000 g·mol⁻¹, more particularly at least 17 000 g·mol⁻¹, even more particularly at least 20 000 g·mol⁻¹ and even more particularly at least 25 000 g·mol⁻¹.

According to a more particular aspect, the gelling agents and/or thickeners present in the composition (C_{D}) that is a subject of the present invention are chosen from cellulose derivatives.

In the context of the present invention, the term “cellulose derivatives” denotes the elements of the group consisting of hydroxyethylcellulose, methylcellulose, ethylcellulose, methylhydroxyethylcellulose, methylhydroxypropylcellulose, hydroxypropylcellulose, the sodium salt of carboxymethylcellulose, and cellulose dihydroxypropyl ether (as described in the American patent published under the number U.S. Pat. No. 4,096,326).

In the context of the present invention, the term “starch” denotes a mixture of amylose and amylopectin, and more particularly the elements of the group consisting of corn starch, wheat starch, potato starch and cassava starch.

According to a particular aspect, the term “linear or branched or crosslinked polymers of polyelectrolyte type” denotes, for the purposes of the present invention:
crosslinked synthetic anionic copolymers based on methacrylic acid or acrylic acid, or esters of methacrylic acid or of acrylic acid, which are optionally hydrophically modified, prepared by direct emulsion polymerization. These synthetic anionic copolymers are known, respectively, to those skilled in the art under the names “Alkaline Swellable Emulsion” (or “ASE”) and “Hydrophobically Alkaline Swellable Emulsion” (or “HASE”). Thickeners of HASE type are described in the international patent application published under the number WO 02/34793 A2;
crosslinked or branched synthetic anionic polyelectrolytes, which are crosslinked and/or branched homopolymers or copolymers of water-soluble unsaturated monomers, such as acrylic acid and/or derivatives thereof, methacrylic acid and/or derivatives thereof, acrylamide and/or derivatives thereof, 2-acrylamido-2-methylpropanesulfonic acid and/or salts thereof, N-vinylpyrrolidone, vinyl alcohol and/or derivatives thereof. These crosslinked or branched synthetic anionic polyelectrolytes are in the form of reverse latices, obtained by reverse emulsion radical polymerization, or in the form of powders, obtained by precipitating polymerization, or by atomization of reverse latices.

According to a particular aspect, the gelling agents and/or thickeners present in the composition (C_{D}) that is a subject of the present invention are chosen from linear or branched or crosslinked polyelectrolytes, obtained from the radical polymerization of at least one monomer selected from the elements of the group consisting of acrylic acid and/or the sodium salt thereof, methacrylic acid and/or the sodium salt thereof, 2-hydroxyethyl acrylate, 2-hydroxyethyl methacrylate, acrylamide, N,N-dimethylacrylamide, N-isopropylacrylamide, 2-acrylamido-2-methylpropanesulfonic acid and/or the sodium or the potassium salt thereof, N-vinylpyrrolidone, in the presence of a crosslinking agent chosen from polyethylenic monomers comprising at least two ethylenic functions, and more particularly chosen from the elements of the group consisting of ethylene glycol dimethacrylate, tetraallyloxyethane, ethylene glycol diacrylate, diallylurea, triallylamine, trimethylolpropane triacrylate or methylenebis(acrylamide), or a mixture of these compounds.

According to a particular aspect, the gelling agents and/or thickeners present in the composition (C_{D}) that is a subject of the present invention are chosen from the elements of the group consisting of:
partially or totally salified acrylic acid homopolymer, crosslinked with triallylamine and/or with trimethylolpropane triacrylate and/or with methylenebis(acrylamide),
the homopolymer of the sodium salt of 2-acrylamido-2-methylpropanesulfonic acid, crosslinked with triallylamine and/or with trimethylolpropane triacrylate and/or with methylenebis (acryl amide),
the copolymer of the sodium salt of 2-acrylamido-2-methylpropanesulfonic acid and of partially or totally salified acrylic acid, crosslinked with triallylamine and/or with trimethylolpropane triacrylate and/or with methylenebis(acrylamide),
the copolymer of the sodium salt of 2-acrylamido-2-methylpropanesulfonic acid and of 2-hydroxyethyl acrylate, crosslinked with triallylamine and/or with trimethylolpropane triacrylate and/or with methylenebis(acrylamide),
the copolymer of the sodium salt of 2-acrylamido-2-methylpropanesulfonic acid and of acrylamide, crosslinked with triallylamine and/or with trimethylolpropane triacrylate and/or with methylenebis(acrylamide),
the terpolymer of the sodium salt of 2-acrylamido-2-methylpropanesulfonic acid, of acrylamide and of partially or totally salified acrylic acid, crosslinked with triallylamine and/or with trimethylolpropane triacrylate and/or with methylenebis(acrylamide),
the terpolymer of the sodium salt of 2-acrylamido-2-methylpropanesulfonic acid, of N,N-dimethylacrylamide and of partially or totally salified acrylic acid, crosslinked with triallylamine and/or with trimethylolpropane triacrylate and/or with methylenebis(acrylamide).

According to a more particular aspect, the gelling agents and/or thickeners present in the composition (C_{D}) that is a subject of the present invention are chosen from the elements of the group consisting of xanthan gum (G_{X}), acacia gum exudate (G_{A}), the mixture of xanthan gum (G_{X}) and of acacia gum exudate (G_{A}) in a mass ratio between the xanthan gum (G_{X}) and the acacia gum exudate (G_{A}) which is greater than or equal to 1/3 and less than or equal to 3/1, the copolymer of the sodium salt of 2-acrylamido-2-methylpropanesulfonic acid and of 2-hydroxyethyl acrylate, crosslinked with triallylamine and/or with trimethylolpropane triacrylate and/or with methylenebis(acrylamide), the copolymer of the sodium salt of 2-acrylamido-2-methylpropanesulfonic acid and of acrylamide, crosslinked with triallylamine and/or with trimethylolpropane triacrylate and/or with methylenebis(acrylamide).

For the purposes of the present invention, the term “contamination” means the penetration into a living organism of substances, called “contaminants”, which alter the biological functions and reactions occurring in said organism.

A distinction is made between:
toxic contamination for which the contaminants are non-living toxic products, such as for example chemical substances or compositions which damage human and/or animal skin and organs, or else radioactive products, and
biological contamination for which the contaminants are pathogenic microorganisms.

These contaminants can be deposited on a solid surface, on a human being or animal, and generally in the environment (on plants, in the subsoil, etc.).

The term “solid surfaces” denotes, for example, floors, walls, window panes, tiles, household electrical appliances, kitchenware, countertops, tapware, sinks, tanks for storing chemical, food or agricultural products, vehicles (motor vehicles, motorbikes, trucks, etc.), pipes or piping, an agricultural machine, a vehicle, a cold room, a kitchen, sanitary ware, an aircraft, a rail car. In general, the term “solid surfaces” denotes object surfaces as opposed to human or animal skin which is excluded from such a definition.

The materials constituting these solid surfaces are, for example, glass (soda-lime, fluorocalcium, borosilicate, crystal), concrete, cemented services, brick, porcelain, earthenware, ceramic, medical devices, polycarbonate or polypropylene plastics, metals, such as for example iron, stainless steel, silver, copper, aluminum, wood, synthetic resins, glass-ceramic or linoleum, and may be coated with paints or varnishes.

In the context of the present invention, the expression “decontamination agent present in the composition (C_{D}) that is a subject of the present invention”, is understood to mean a chemical compound or a chemical composition which makes it possible, after application to the solid surface to be treated, to reduce the amount of contaminating substances present or else to eliminate them from said solid surface.

The decontamination agent is chosen according to the use for which the composition (C_{D}) is intended, in particular as a function of the nature of the contamination and of the solid surface to be decontaminated.

In the context of the present invention, the term “disinfectant” denotes a chemical substance or composition which kills or inactivates microorganisms present on solid surfaces.

The disinfectants included in the composition (C_{D}) which is a subject of the present invention belong to biocidal products as defined by the regulations concerning the making available on the market and the use of biocidal products (EU Regulation No. 528/2012 of May 22, 2012).

Biocidal products represent all substances and mixtures, consisting of one or more active molecules, intended to destroy, repel or render harmless living pest organisms, to prevent their action or to combat them in any other way by chemical or biological action. These products are divided, depending on their applications, into four groups which are (i) disinfectants, (ii) protective products aimed at preventing microbial growth and algal growth, (iii) pest control products and (iv) other biocidal products such as anti-fouling products or products for embalming and taxidermy.

The effectiveness of disinfectants depends on their spectrum of action on the different types of biological agents. Bactericidal agents (action on bacteria), fungicidal agents (action on fungi), virucidal agents (action on viruses) and sporicidal agents (action on spores) are defined in this way. In addition, each disinfectant has several performance criteria, such as (i′) its speed of efficiency, (ii′) its decontamination efficiency which is measured by a factor of reduction of an initial contaminating population under the effect of the disinfectant (initial population/final population after treatment) or by the log reduction of this factor, and (iii′) its compatibility with construction materials. Disinfectants are therefore classified according to their disinfection efficiency and references are made to disinfectants with a high, medium or low level of disinfection.

The composition (C_{D}) that is a subject of the present invention comprises at least one disinfectant chosen from disinfectants with a high level of disinfection, namely disinfectants exhibiting a factor (initial contaminating population/final population after treatment) of greater than 10⁶.

The chlorinated products used as disinfectants in the composition (C_{D}) that is a subject of the present invention are characterized by their wide spectrum of activity since they are bactericidal, virucidal, fungicidal and sporicidal. Their action time is fast and equal to their drying time.

According to a more particular aspect, the composition (C_{D}) that is a subject of the present invention comprises at least one disinfectant which is a chlorinated product chosen from the elements of the group consisting of chlorine, sodium hypochlorite (bleach) and chlorine dioxide.

According to an even more particular aspect, the composition (C_{D}) that is a subject of the present invention comprises a disinfectant which is sodium hypochlorite (bleach).

The aldehydes used as disinfectants in the composition (C_{D}) that is a subject of the present invention are characterized by their wide spectrum of activity since they are bactericidal, fungicidal, virucidal and sporicidal. They are characterized by a mode of action aimed at causing denaturation of nucleic acids and proteins in the microorganisms.

According to a more particular aspect, the composition (C_{D}) that is a subject of the present invention comprises at least one disinfectant which is an aldehyde chosen from the elements of the group consisting of glutaraldehyde and succinic aldehyde.

The oxidizing agents used as disinfectants in the composition (C_{D}) that is a subject of the present invention are characterized by a wide spectrum of activity since they are bactericidal, virucidal, fungicidal and sporicidal. They are characterized by a mode of action aimed at destroying organic membranes.

According to a more particular aspect, the composition (C_{D}) that is a subject of the present invention comprises at least one disinfectant which is an oxidizing agent chosen from the elements of the group consisting of hydrogen peroxide; activated peroxides such as mixtures of hydrogen peroxide and sodium bicarbonate, mixtures of hydrogen peroxide and urea, mixtures of hydrogen peroxide and peracetic acid, and mixtures of hydrogen peroxide and iron (Fenton's reagent); hydroperoxycarbonates; peracetic acid; sodium perborate; sodium percarbonate, optionally perhydrated; sodium peroxysilicate; sodium peroxypyrophosphate; sodium peroxysilicate and aryloxides such as arylbenzenesulfonates.

According to an even more particular aspect, the composition (C_{D}) that is a subject of the present invention comprises at least one disinfectant which is chosen from the elements of the group consisting of hydrogen peroxide and mixtures of hydrogen peroxide and peracetic acid.

According to an even more particular aspect, the composition (C_{D}) that is a subject of the present invention comprises a disinfectant which is hydrogen peroxide.

According to a more particular aspect, the composition (C_{D}) that is a subject of the present invention comprises at least one decontamination agent which is chosen from the elements of the group consisting of sorbic acid, potassium sorbate, sodium sorbate, dehydroacetic acid, sodium dehydroacetate, benzoic acid, sodium benzoate and potassium benzoate.

According to another even more particular aspect, the composition (C_{D}) that is a subject of the present invention comprises at least one decontamination agent which is a mixture of potassium sorbate and sodium benzoate.

According to a more particular aspect, the composition (C_{D}) that is a subject of the present invention comprises at least one decontamination agent which is a basic agent chosen from the elements of the group consisting of sodium hydroxide and potassium hydroxide.

According to another particular aspect, a subject of the invention is the aqueous composition composition (C_{D}) as defined above wherein the at least one decontamination agent is an acidic agent chosen from the group consisting of sorbic acid, potassium sorbate, sodium sorbate, dehydroacetic acid, sodium dehydroacetate, benzoic acid, sodium benzoate and potassium benzoate.

According to another even more particular aspect, a subject of the invention is the aqueous composition composition (C_{D}) as defined above wherein the at least one decontamination agent is an acidic agent which is a mixture of potassium sorbate and sodium benzoate.

The aqueous composition (C_{D}) that is a subject of the present invention may optionally comprise additional ingredients that are usually found in compositions intended for the decontamination of a solid surface, for example inorganic salts, salts of organic compounds, surfactants, solvents, antioxidants or anticorrosion agents.

As examples of solvents that are optionally present in the aqueous composition (C_{D}) that is a subject of the present invention, examples that may be mentioned include ethanol, 1-propanol, 2-propanol, 1-butanol, polyhydric alcohols such as glycerol, diglycerol, triglycerol, glycerol oligomers, xylitol, erythritol, sorbitol, 2-methyl-1,3-propanediol; alkoxylated polyhydric alcohols; glycols such as butylene glycol, hexylene glycol, caprylyl glycol or 1,2-octanediol or 1,2-pentanediol, pentylene glycol, monopropylene glycol, dipropylene glycol, isoprene glycol, butyl diglycol, trimethyl trimethylene glycol, polyethylene glycols with a molecular weight of 200 g·mol⁻¹ and 8000 g·mol⁻¹.

According to a particular aspect, per 100% by mass of the composition (C_{D}), the solvents as described above may constitute a mass proportion of greater than or equal to 0.5% and less than or equal to 10%, more particularly greater than or equal to 0.5% and less than or equal to 5%, greater than or equal to 0.5% and less than or equal to 3%, greater than or equal to 0.8% and less than or equal to 3%.

The term “inorganic salts” denotes heteropolar compounds, the crystal lattice of which comprises the participation of at least one type of cation other than the hydrogen ion and of at least one type of anion other than the hydroxide ion, for instance salts consisting of a cation which is the ammonium ion or a metal cation and of an anion selected from the elements of the group consisting of halides, carbonates, bicarbonates, phosphates, nitrates, borates and sulfates, and more particularly sodium, magnesium or calcium chloride; aluminum, barium, ammonium or zinc sulfate heptahydrate, potassium aluminum sulfate; aluminum, manganese or zinc borate; strontium or potassium tetraborate hydrate; sodium, aluminum, zinc, manganese or magnesium dihydrogen phosphate, monocalcium, monopotassium, zinc, manganese or aluminum phosphate, copper, iron, nickel, manganese, zinc, beryllium, cerium, calcium, lithium, cobalt, chromium, zirconium, strontium or potassium carbonate or sodium carbonate hexahydrate; calcium or strontium hydrogen phosphate; disodium, dimanganese or dizinc hydrogen phosphate; potassium, sodium, calcium or magnesium bicarbonate; iron, titanium, zinc or antimony oxide; iron ammonium sulfate, magnesium ammonium phosphate; magnesium hydrogen sulfate; strontium, barium or magnesium metaborate hydrate; magnesium trisilicate, sodium, zirconium or magnesium nitrate, potassium metaphosphate, potassium tripolyphosphate, sodium trimetaphosphate, ammonium molybdate, ammonium octamolybdate or ammonium heptamolybdate.

The term “salts of organic compounds” denotes a salt consisting of a cation which is the ammonium ion or a metal cation and of an organic anion which is an organic compound bearing at least one carboxylic acid function in carboxylate form or at least one sulfonic acid function in sulfonate form or at least one sulfate function, for example ammonium, sodium, copper, magnesium or potassium citrate; calcium, copper, sodium, potassium, strontium or zinc acetate; sodium, ammonium, aluminum, manganese or potassium oxalate.

As examples of anticorrosion agents and/or antioxidants that are optionally present in the composition (C_{D}) that is a subject of the present invention, examples that may be mentioned include urea, alkanolamines, sodium and/or potassium and/or calcium salts of organic acids, for instance sodium lactate, sodium citrate, sodium gluconate, sodium ascorbate, sodium succinate, and/or inorganic salts, for instance sodium nitrite, sodium molybdate, sodium phosphates and/or polyphosphates and/or any other compound known to those skilled in the art.

Among the surfactants which are optionally present in the composition (C_{ED}), there are anionic, cationic or amphoteric surfactants. As examples of anionic surfactants, examples that may be mentioned include alkyl ether sulfates, alkyl sulfates, alkylamido ether sulfates, alkylaryl polyether sulfates, monoglyceride sulfates, α-olefin sulfonates, paraffin sulfonates, alkyl phosphates, alkyl ether phosphates, alkyl sulfonates, alkylamidesulfonates, alkylarylsulfonates, alkyl carboxylates, alkylsulfosuccinates, alkyl ether sulfosuccinates, alkylamide sulfosuccinates, alkylsulfoacetates or acyllactylates of alkali metals, of alkaline-earth metals, of ammonium, of amines or of amino alcohols.

As examples of amphoteric surfactants that are optionally present in the composition (C_{D}) that is a subject of the present invention, examples that may be mentioned include alkylbetaines, alkylamidobetaines, sultaines, alkylamidoalkylsulfobetaines, imidazoline derivatives, phosphobetaines, amphopolyacetates and amphopropionates.

As examples of cationic non-fluoro surfactants that are optionally present in the composition (C_{D}) that is a subject of the present invention, examples that may be mentioned include quaternary ammonium derivatives.

A subject of the invention is also a process for decontaminating a solid surface, characterized in that it comprises:
at least one step A₁ of preparing a foam obtained by mixing a composition (C_{D}) according to the invention with a gas or a mixture of gases, chosen from air, nitrogen or carbon dioxide, followed by
at least one step A₂ of bringing the foam prepared in step A₁ into contact with the solid surface.

The foam prepared in step A₁ of the process that is a subject of the present invention is generated by any foam-generating system known to those skilled in the art and described in the prior art, for instance mechanical stirring of the aqueous composition (C_{D}), bubbling of one or more gases into the aqueous composition (C_{D}), the use of a static ball mixer or any other device for ensuring mixing between the gas or the mixture of gases and the aqueous composition (C_{D}), or else a device using a projection or spray nozzle.

The foam prepared in step A₁ of the process that is a subject of the present invention may be produced by various items of equipment known to those skilled in the art, for instance injectors, lances, generators or spouts.

According to a particular aspect, a subject of the invention is the process as defined above, characterized in that step A₁ consists of a step A₁′ of diluting said composition (C_{D}) with water, in a (C_{D})/water volume ratio of between 10/90 and 1/99, followed by a step A₁″ of mixing the dilute composition obtained at the end of step A₁′ with a gas or a mixture of gases chosen from air, nitrogen or carbon dioxide.

Step A₁′ of dilution of the aqueous composition (C_{D}) with water may be performed via any means known to those skilled in the art, for instance in tanks of suitable dimensions and with stirring at a suitable rate. More particularly, the water of dilution and the aqueous composition (C_{D}) may be transported separately, and then conveyed to a generator which creates the foam by mixing the water of dilution and the composition (C_{D}) with air, and then sprays it, at variable and adjustable flow rates, toward the contaminated surface to be treated. In such a case, the mixing of water of dilution and of the aqueous composition (C_{D}) with air is performed by air suction during the expansion of the dilute foam solution.

In the process of the invention, step A₂ of bringing into contact the foam created during step A₁) or step A₁″ is performed by spraying onto the contaminated surface to be treated, via means known to those skilled in the art, for instance a lance, a generator or a foam cannon.

According to one particular aspect, a subject of the invention is the process as defined above, characterized in that it also comprises a step A₃ of rinsing the solid surface obtained at the end of step A₂.

Step A₃ of rinsing said solid surface can be carried out by means of a rinsing solution or a rinsing foam or water.

The rinsing foam or the rinsing solution can be any suitable foam or any suitable solution, depending on the nature of the decontamination foam used during step A₂ and/or depending on the nature of the solid surface to be rinsed. It may be:
a rinsing foam comprising a mixture of water, of the surfactant mixture (M₁) as described above, and of a gelling agent (AG) as described above, optionally comprising a buffer compound, the nature of which depends on the identity of the decontamination agent used in the composition (C_{D}) that is a subject of the invention,
a rinsing foam comprising water, and optionally a buffer compound, the nature of which depends on the identity of the decontamination agent used in the composition (C_{D}) that is a subject of the invention.

In rinsing step A₃, if the decontamination agent used in the composition (C_{D}) is an acidic agent, a rinsing foam or a rinsing solution comprising a buffer of basic nature will be chosen; in rinsing step A₃, if the decontamination agent used in the composition (C_{D}) is a basic agent, a rinsing foam or a rinsing solution comprising a buffer of acidic nature will be chosen.

### DESCRIPTION OF THE PREFERRED EMBODIMENTS

The examples that follow illustrate the invention without, however, limiting it.

### 1) Preparation of Foaming Compositions

### 1.1) Preparation of a Solution of Disodium N-cocoyl Glutamate [Composition (C₁)]

375 kg of water and 250 kg of monosodium sodium glutamate monohydrate are placed in a reactor, with stirring and at a temperature of 20° C., followed by 184 kg of an aqueous sodium hydroxide solution at 30% by mass so as to reach a pH of 12. 245 kg of cocoyl chloride, which is a mixture of acid chlorides comprising, per 100% by mass, 8% by mass of octanoyl chloride, 8% by mass of decanoyl chloride, 50% by mass of lauroyl chloride, 17% by mass of myristoyl chloride, 8% by mass of palmitoyl chloride, 3% by mass of stearoyl chloride, 4% by mass of oleoyl chloride and 2% by mass of linoleoyl chloride, are then added gradually with stirring, followed by a further 140 kg of the 30% sodium hydroxide solution to keep the pH between 11 and 12. The temperature is maintained between 20° C. and 50° C. for 2 hours.

The mixture obtained is acidified by adding 54 kg of an aqueous sulfuric acid solution at 70% by mass, and is then diluted with 193 kg of water to obtain an aqueous solution of disodium N-cocoyl glutamate [composition (C₁)].

### 1.2) Analytical Features of the Solutions Prepared Previously

The analytical features of composition (C₁) are collated in table 1 below.

TABLE 1

| | (C₁) |
|---|---|
| Appearance at 20° C. (visual method) | Clear |
| Mass content of water (a) (according to | 68.00% |
| the standard NFT 73-201) | |
| Residual fatty acids (b) (gas | 5.8% |
| chromatography (GC)) | |
| pH | 6.3 |
| Sodium chloride content (potentiometric | 3.60% |
| titration (c)) | |
| Sodium sulfate content (d) (calculated | 5.20% |
| on feedstock) | |
| Citrate content (e) (calculated on feedstock) | 0% |
| Mass content of active material | 17.40% |
| (AM₁) (cocoyl glutamate) | |
| (AM₁) = 100% − (a) − (b) − (c) − (d) − (e) | |

### 2) Preparation of Alkylpolyglycoside-Based Surfactant Compositions

### 2.1) Preparation of a Composition (C₃)

3.7 molar equivalents of a mixture of fatty alcohols (N₁) consisting, per 100% of its mass, of 68% by mass of 1-dodecanol, of 25% by mass of 1-tetradecanol and of 7% by mass of 1-hexadecanol, and then 1 molar equivalent of anhydrous glucose are poured with stirring into a reactor maintained at 80° C., followed by 0.15% by mass of 98% sulfuric acid per 100% by mass of the mixture.

The reaction medium is placed under a partial vacuum of about 0.18×10⁵ Pa (180 mbar) and maintained at 100° C.-105° C. for 4 hours with distillation of the water formed.

After cooling to 85° C.-90° C. and neutralizing by addition of 40% sodium hydroxide, the reaction medium thus obtained is discharged at 70° C. and filtered to remove the grains of unreacted glucose.

The filtrate is then poured into another reactor and the excess of the mixture of fatty alcohols (N₁) is removed by distillation using a thin-film evaporator, and the residue is then diluted in water. After stirring for 30 minutes at 50° C., composition (C₃) is obtained, which comprises 50% by mass of water and 50% by mass of a mixture of alkylpolyglucosides (AM_{APG1}), for which the proportions of alkylpolyglucosides and the mean degree of polymerization of their polyglucoside residue are determined by gas chromatography (GC); it thus comprises, per 100% by mass, 69% by mass of n-dodecyl polyglucosides, 25% by mass of n-tetradecyl polyglucosides and 6% by mass of n-hexaldecyl polyglucosides with a degree of polymerization equal to 1.25.

2.2) Preparation of a Composition (Ca) Comprising n-heptyl Polyglucoside

2.7 molar equivalents of 1-heptanol and then 1 molar equivalent of anhydrous glucose are poured with stirring into a reactor maintained at 40° C., followed by 0.15% by mass of 98% sulfuric acid per 100% by mass of the mixture.

The reaction medium is placed under a partial vacuum of about 0.18×10⁵ Pa (180 mbar) and maintained at 100° C.-105° C. for 4 hours with distillation of the water formed. After cooling to 85° C.-90° C. and neutralizing by addition of 40% sodium hydroxide, the reaction medium thus obtained is discharged at 70° C. and filtered to remove the grains of unreacted glucose.

The filtrate is then poured into another reactor and the excess heptanol is distilled off under partial vacuum, and the residue is then diluted in water.

After stirring for 30 minutes at 50° C., composition (C₄) is obtained comprising 26.4% by mass of water and 73.6% by mass of n-heptyl polyglucosides (AM_{APG2}), with a degree of polymerization, determined by GC, equal to 1.25.

2.3) Preparation of a Composition (C₅) Comprising n-Octyl Polyglucoside and n-decyl Polyglucoside

2.7 molar equivalents of a mixture of fatty alcohols (N₃) consisting, per 100% of its mass, of 50% by mass of 1-octanol and of 50% by mass of 1-decanol, and then 1 molar equivalent of anhydrous glucose are poured with stirring into a reactor maintained at 80° C., followed by 0.15% by mass of 98% sulfuric acid per 100% by mass of the mixture.

The reaction medium is placed under a partial vacuum of about 0.18×10⁵ Pa (180 mbar) and maintained at 100° C.-105° C. for 4 hours with distillation of the water formed.

After cooling to 85° C.-90° C. and neutralizing by addition of 40% sodium hydroxide, the reaction medium thus obtained is discharged at 70° C. and filtered to remove the grains of unreacted glucose.

The filtrate is then poured into another reactor and the excess of the mixture of fatty alcohols (N₃) is removed by distillation using a thin-film evaporator, and the residue is then diluted in water.

After stirring for 30 minutes at 50° C., composition (C₅) is obtained, which comprises 40% by mass of water and 60% by mass of a mixture of alkylpolyglucosides (AM_{APG3}), for which the proportions of alkylpolyglucosides and the mean degree of polymerization of their polyglucoside residue are determined by GC; it thus comprises, per 100% by mass, 52% by mass of n-octyl polyglucoside and 48% by mass of n-decyl polyglucoside, with a degree of polymerization equal to 1.30.

### 2.4) Preparation of Compositions Based on Disodium Cocoylglutamate and Alkylpolylglucosides

2.4.1) Preparation of a Composition (T₁) Comprising Disodium N-cocoylglutamate [Composition (C₁)], a Mixture of n-dodecyl Polyglucosides, n-tetradecyl Polyglucosides and n-hexadecyl Polyglucosides [Composition (C₃)], and n-heptyl Polyglucoside [Composition (C₄)]

A composition (T₁) is prepared by pouring, with stirring, the composition (C₁) and the compositions (C₃) and (C₄) into a reactor maintained at 40° C. The mixture is stirred for thirty minutes in order to obtain the composition (T₁). The amounts used are listed in table 2 below.

2.4.2) Preparation of a Composition (T₂) Comprising Disodium N-cocoylglutamate [Composition (C₁)], a Mixture of n-dodecyl Polyglucosides, n-tetradecyl Polyglucosides and n-hexadecyl Polyglucosides [Composition (C₃)]

A composition (T₂) is prepared by pouring, with stirring, the composition (C₁) and the composition (C₃) into a reactor maintained at 40° C. The mixture is stirred for thirty minutes in order to obtain the composition (T₂). The amounts used are listed in table 2 below.

TABLE 2
Amounts used

| | (C₁) | (C₃) | (C₄) |
|---|---|---|---|
| (T₁) | 74.9 g | 11.1 g | 14.0 g |
| (T₂) | 93.0 g | 7.0 g | 0 g |

The analytical features of the compositions (T₁) and (T₂′) are collated in table 1 below.

TABLE 3

| | AG⁽¹⁾ | Δ⁽²⁾ | T′⁽³⁾ | H₂O (%) | Δ₁⁽⁴⁾ | AMC⁽⁵⁾ as % | Appearance |
|---|---|---|---|---|---|---|---|
| (T₁) | 5.84% | 0.45 | 0.55 | 60.15% | 0.54 | 28.85% | Homogeneous |
| (T₂) | 5.4% | 4.6 | 0.18 | 66.74% | — | 19.68% | Homogeneous |

⁽¹⁾Residual fatty acids (mass percentage)
⁽²⁾Δ = (AM₁)/[(AM_{APG1}) + (AM_{APG2})]
⁽³⁾T' = [(AM_{APG1}) + (AM_{APG2})]/[(AM₁) + (AM_{APG1}) + (AM_{APG2})]
⁽⁴⁾Δ₁ = (AM_{APG1})/(AM_{APG2})
⁽⁵⁾AMC = [(AM₁) + (AM_{APG1}) + (AM_{APG2})]/total mass, with total mass = 100 g.

### 3) Preparation of Compositions According to the Invention and of Comparative Compositions

3.1) Preparation of Compositions According to the Invention E₁ to E₆ and of a Comparative Composition E₀

The compositions E₀ to E₆ are prepared at a temperature of 25° C., in a reactor of suitable volume fitted with anchor-type mechanical stirring at a speed of 50 revolutions·min⁻¹. The ingredients are gradually introduced one after the other until a homogeneous and liquid composition is obtained.

The compositions are described in detail in table 4 below:

TABLE 4

| | E₀ | E₁ | E₂ | E₃ | E₄ |
|---|---|---|---|---|---|
| Composition (T₁) | 2.36%, i.e. | 2.36%, i.e. | 2.36%, i.e. | 2.36%, i.e. | 2.36%, i.e. |
| at 28.85% of | 0.68% of | 0.68% of | 0.68% of | 0.68% of | 0.68% of |
| AMC⁽⁵⁾ | AMC⁽⁵⁾ | AMC⁽⁵⁾ | AMC⁽⁵⁾ | AMC⁽⁵⁾ | AMC⁽⁵⁾ |
| Xanthan solution | 15% | 15% | 15% | 15% | 15% |
| at 1.66% by mass | | | | | |
| Deionized water | q.s. 100% | q.s. 100% | q.s. 100% | q.s. 100% | q.s. 100% |
| Bleach at 12° | — | 7.1% by | 21.4% by | — | — |
| chlorine | | mass of | mass of | | |
| | | NaOCl | NaOCl | | |
| Aqueous | — | — | — | 3.3% by | 16.7% by |
| hydrogen peroxide | | | | mass of | mass of |
| solution at | | | | H₂O₂ | H₂O₂ |
| 35% by mass | | | | | |
| pH produced | 6.5 | 6.4 | 6.7 | 6.4 | 6.2 |
| Visual | Homogeneous | Homogeneous | Homogeneous | Homogeneous | Homogeneous |
| appearance | liquid | liquid | liquid | liquid | liquid |
| produced | | | | | |

TABLE 4 (a)

| | E₅ | E₆ |
|---|---|---|
| Composition (T₁) | 2.36%, i.e. | 2.36%, i.e. |
| at 28.85% of | 0.68% of | 0.68% of |
| AMC⁽⁵⁾ | AMC⁽⁵⁾ | AMC⁽⁵⁾ |
| Xanthan solution | 15% | 15% |
| at 1.66% by mass | | |
| Deionized water | q.s. 100% | q.s. 100% |
| Euxyl ™ K712⁽⁶⁾ | 5% | 10% |
| pH produced | 6.5 | 6.4 |
| Visual | Homogeneous | Homogeneous |
| appearance | liquid | liquid |
| produced | | |

⁽⁶⁾Euxyl ™ K712: aqueous solution containing 45% of active material, said active material comprising, per 100% of its mass, 33% by mass of potassium sorbate and 67% by mass of sodium benzoate.

3.2) Preparation of Comparative Compositions F₀ to F₄, Comprising the Composition (T₂)

The compositions F₀ to F₄ are prepared according to the procedure described in section 3.1 above.

The compositions F₀ to F₄ are described in detail in table 5 below:

TABLE 5

| | F₀ | F₁ | F₂ | F₃ | F₄ |
|---|---|---|---|---|---|
| Composition (T₂) | 3.45%, i.e. | 3.45%, i.e. | 3.45%, i.e. | 3.45%, i.e. | 3.45%, i.e. |
| at 19.68% of | 0.68% of | 0.68% of | 0.68% of | 0.68% of | 0.68% of |
| AMC⁽⁵⁾ | AMC⁽⁵⁾ | AMC⁽⁵⁾ | AMC⁽⁵⁾ | AMC⁽⁵⁾ | AMC⁽⁵⁾ |
| Xanthan solution | 15% | 15% | 15% | 15% | 15% |
| at 1.66% by mass | | | | | |
| Deionized water | q.s. 100% | q.s. 100% | q.s. 100% | q.s. 100% | q.s. 100% |
| Bleach at 12° | — | 7.1% by | 21.4% by | — | — |
| chlorine | | mass of | mass of | | |
| | | NaOCl | NaOCl | | |
| Aqueous | — | — | — | 3.3% by | 16.7% by |
| hydrogen peroxide | | | | mass of | mass of |
| solution at | | | | H₂O₂ | H₂O₂ |
| 35% by mass | | | | | |
| pH produced | 6.4 | 6.4 | 6.5 | 6.2 | 6.0 |
| Visual | Homogeneous | Homogeneous | Homogeneous | Homogeneous | Homogeneous |
| appearance | liquid | liquid | liquid | liquid | liquid |
| produced | | | | | |

3.3) Preparation of Comparative Compositions F₅ to F₉, Comprising the Composition (C₁)

The compositions F₅ to F₉ are prepared according to the procedure described in section 3.1 above.

The compositions F₅ to F₉ are described in detail in table 6 below:

TABLE 6

| | F₅ | F₆ | F₇ | F₈ | F₉ |
|---|---|---|---|---|---|
| Composition (C₁) | 3.90%, i.e. | 3.90%, i.e. | 3.90%, i.e. | 3.90%, i.e. | 3.90%, i.e. |
| at 17.4% of | 0.68% of | 0.68% of | 0.68% of | 0.68% of | 0.68% of |
| AM1 | AM1 | AM1 | AM1 | AM1 | AM1 |
| Xanthan solution | 15% | 15% | 15% | 15% | 15% |
| at 1.66% by mass | | | | | |
| Deionized water | q.s. 100% | q.s. 100% | q.s. 100% | q.s. 100% | q.s. 100% |
| Bleach at 12° | — | 7.1% by | 21.4% by | — | — |
| chlorine | | mass of | mass of | | |
| | | NaOCl | NaOCl | | |
| Aqueous | — | — | — | 3.3% by | 16.7% by |
| hydrogen peroxide | | | | mass of | mass of |
| solution at | | | | H₂O₂ | H₂O₂ |
| 35% by mass | | | | | |
| pH produced | 6.3 | 6.8 | 6.7 | 6.5 | 6.2 |
| Visual | Homogeneous | Homogeneous | Homogeneous | Homogeneous | Homogeneous |
| appearance | liquid | liquid | liquid | liquid | liquid |
| produced | | | | | |

3.4) Preparation of Comparative Compositions F₁₀ to F₁₄, Comprising the Composition (C₃)

The compositions F₁₀ to F₁₄ are prepared according to the procedure described in section 3.1 above.

The compositions F₁₀ to F₁₄ are described in detail in table 7 below:

TABLE 7

| | F₁₀ | F₁₁ | F₁₂ | F₁₃ | F₁₄ |
|---|---|---|---|---|---|
| Composition (C₃) | 1.36%, i.e. | 1.36%, i.e. | 1.36%, i.e. | 1.36%, i.e. | 1.36%, i.e. |
| at 50.0% of | 0.68% of | 0.68% of | 0.68% of | 0.68% of | 0.68% of |
| AM_{APG1} | AM_{APG1} | AM_{APG1} | AM_{APG1} | AM_{APG1} | AM_{APG1} |
| Xanthan solution | 15% | 15% | 15% | 15% | 15% |
| at 1.66% by mass | | | | | |
| Deionized water | q.s. 100% | q.s. 100% | q.s. 100% | q.s. 100% | q.s. 100% |
| Bleach at 12° | — | 7.1% by | 21.4% by | — | — |
| chlorine | | mass of | mass of | | |
| | | NaOCl | NaOCl | | |
| Aqueous | — | — | — | 3.3% by | 16.7% by |
| hydrogen peroxide | | | | mass of | mass of |
| solution at | | | | H₂O₂ | H₂O₂ |
| 35% by mass | | | | | |
| pH produced | 6.4 | 6.6 | 6.8 | 6.9 | 6.1 |
| Visual | Homogeneous | Homogeneous | Homogeneous | Homogeneous | Homogeneous |
| appearance | liquid | liquid | liquid | liquid | liquid |
| produced | | | | | |

3.5) Preparation of Comparative Compositions F₁₅ to F₁₉, Comprising the Composition (C₄)

The compositions F₁₅ to F₁₉ are prepared according to the procedure described in section 3.1 above.

The compositions F₁₅ to F₁₉ are described in detail in table 8 below:

TABLE 8

| | F₁₅ | F₁₆ | F₁₇ | F₁₈ | F₁₉ |
|---|---|---|---|---|---|
| Composition (C₄) | 0.92%, i.e. | 0.92%, i.e. | 0.92%, i.e. | 0.92%, i.e. | 0.92%, i.e. |
| at 73.6% of | 0.68% of | 0.68% of | 0.68% of | 0.68% of | 0.68% of |
| AM_{APG2} | AM_{APG2} | AM_{APG2} | AM_{APG2} | AM_{APG2} | AM_{APG2} |
| Xanthan solution | 15% | 15% | 15% | 15% | 15% |
| at 1.66% by mass | | | | | |
| Deionized water | q.s. 100% | q.s. 100% | q.s. 100% | q.s. 100% | q.s. 100% |
| Bleach at 12° | — | 7.1% by | 21.4% by | — | — |
| chlorine | | mass of | mass of | | |
| | | NaOCl | NaOCl | | |
| Aqueous | — | — | — | 3.3% by | 16.7% by |
| hydrogen peroxide | | | | mass of | mass of |
| solution at | | | | H₂O₂ | H₂O₂ |
| 35% by mass | | | | | |
| pH produced | 6.3 | 6.5 | 6.5 | 6.4 | 6.5 |
| Visual | Homogeneous | Homogeneous | Homogeneous | Homogeneous | Homogeneous |
| appearance | liquid | liquid | liquid | liquid | liquid |
| produced | | | | | |

3.6) Preparation of Comparative Compositions F₂₀ to F₂₄, Comprising the Composition (C₄)

The compositions F₂₀ to F₂₄ are prepared according to the procedure described in section 3.1 above.

The compositions F₂₀ to F₂₄ are described in detail in table 9 below:

TABLE 9

| | F₂₀ | F₂₁ | F₂₂ | F₂₃ | F₂₄ |
|---|---|---|---|---|---|
| Composition (C₅) | 1.13%, i.e. | 1.13%, i.e. | 1.13%, i.e. | 1.13%, i.e. | 1.13%, i.e. |
| at 60.0% of | 0.68% of | 0.68% of | 0.68% of | 0.68% of | 0.68% of |
| AM_{APG3} | AM_{APG3} | AM_{APG3} | AM_{APG3} | AM_{APG3} | AM_{APG3} |
| Xanthan solution | 15% | 15% | 15% | 15% | 15% |
| at 1.66% by mass | | | | | |
| Deionized water | q.s. 100% | q.s. 100% | q.s. 100% | q.s. 100% | q.s. 100% |
| Bleach at 12° | — | 7.1% by | 21.4% by | — | — |
| chlorine | | mass of | mass of | | |
| | | NaOCl | NaOCl | | |
| Aqueous | — | — | — | 3.3% by | 16.7% by |
| hydrogen peroxide | | | | mass of | mass of |
| solution at | | | | H₂O₂ | H₂O₂ |
| 35% by mass | | | | | |
| pH produced | 6.8 | 6.4 | 6.0 | 6.2 | 6.0 |
| Visual | Homogeneous | Homogeneous | Homogeneous | Homogeneous | Homogeneous |
| appearance | liquid | liquid | liquid | liquid | liquid |
| produced | | | | | |

### 4) Evaluation of the Foaming Properties

4.1) Principle of the Evaluation Method

The evaluation of the foaming properties of the test compositions is performed by forming a foam, from a solution of WHO hard water comprising a predetermined mass content of the test compositions, by mechanical stirring at a temperature of 20° C.

4.2) Experimental Protocol

250 cm³ aqueous solutions are prepared so as to obtain solutions at 0.5% by mass of surfactant active material in WHO hard water, from the compositions E₀ to E₄, and F₀ to F₂₄, as described above.

The WHO hard water contains, per liter of deionized water, 0.403 g of anhydrous calcium chloride and 0.139 g of magnesium chloride hexahydrate; which gives it a hardness titer equal to 34° Th.

These solutions are poured into a 500 cm³ beaker and are then stirred using a Rayneri™ laboratory blender (model 33/300) equipped with a butterfly paddle with three hollow arms, at a constant speed of 3000 rpm for 2 minutes.

4.3) Expression of the Results

The following parameters are measured for each test:
The expansion time (Tₑₓₚ): this is the stirring time after which suppression of the vortex in the beaker is observed. Beyond this time, the foam totally surrounds the shaft of the paddle and its level is horizontal;
The half-life time (T_{1/2}): this is the time after which the foam obtained from a certain volume of foaming solution became drained of an amount of solution corresponding to half of the initial volume. For this test, the half-life time is reached when the upper level of the draining water reaches the 125 cm³ mark on the beaker;
The height of foam generated by stirring (Hₜₒ): this is the height of foam generated at the end of the 2 minutes of stirring;
The residual foam height after 30 minutes (Hₜ₃₀): this is the foam height observed 30 minutes after the end of the 2 minutes of stirring.
The difference Δ_{H}=(Hₜₒ−Hₜ₃₀) makes it possible to evaluate comparatively the quality of the foams generated by the various surfactants.

4.4) Results Obtained

The results obtained for the aqueous solutions of active material in WHO hard water for the compositions of the compositions E₀ to E₆, and F₀ to F₂₄ are shown in tables 10 to 15 below.

TABLE 10

| | (Tₑₓₚ) | (T_{1/2}) | (Hₜₒ) | (Hₜ₃₀) | (ΔH) |
|---|---|---|---|---|---|
| E₀ | 18 s | >240 | 125 | 120 | 5 |
| | | min | mm | mm | mm |
| E₁ | 7 s | >240 | 140 | 130 | 10 |
| | | min | mm | mm | mm |
| E₂ | 6 s | >240 | 140 | 130 | 10 |
| | | min | mm | mm | mm |
| E₃ | 17 s | >240 | 140 | 130 | 10 |
| | | min | mm | mm | mm |
| E₄ | 22 s | >240 | 140 | 130 | 10 |
| | | min | mm | mm | mm |
| E₅ | 6 s | >240 | 132 | 130 | 2 |
| | | min | mm | mm | mm |
| E₆ | 8 s | >240 | 134 | 134 | 0 |
| | | min | mm | mm | mm |

TABLE 11

| | (Tₑₓₚ) | (T_{1/2}) | (Hₜₒ) | (Hₜ₃₀) | (ΔH) |
|---|---|---|---|---|---|
| F₀ | 12 s | >240 | <100 | <100 | — |
| | | min | mm | mm | |
| F₁ | 8 s | >240 | <100 | <100 | — |
| | | min | mm | mm | |
| F₂ | 9 s | 150 | <100 | <100 | — |
| | | min | mm | mm | |
| F₃ | 9 s | >240 | <100 | <100 | — |
| | | min | mm | mm | |
| F₄ | 8 s | >240 | <100 | <100 | — |
| | | min | mm | mm | |

TABLE 12

| | (Tₑₓₚ) | (T_{1/2}) | (Hₜₒ) | (Hₜ₃₀) | (ΔH) |
|---|---|---|---|---|---|
| F₅ | >120 s | >240 | 80 | <80 | — |
| | | min | mm | mm | |
| F₆ | 35 s | >240 | 110 | <100 | — |
| | | min | mm | mm | |
| F₇ | >120 s | >240 | <100 | <100 | — |
| | | min | mm | mm | |
| F₈ | >120 s | >240 | <100 | <100 | — |
| | | min | mm | mm | |
| F₉ | >120 s | >240 | <100 | <100 | — |
| | | min | mm | mm | |

TABLE 13

| | (Tₑₓₚ) | (T_{1/2}) | (Hₜₒ) | (Hₜ₃₀) | (ΔH) |
|---|---|---|---|---|---|
| F₁₀ | >120 s | >240 | <100 | <100 | — |
| | | min | mm | mm | |
| F₁₁ | 35 s | >240 | <100 | <100 | — |
| | | min | mm | mm | |
| F₁₂ | >120 s | >240 | <100 | <100 | — |
| | | min | mm | mm | |
| F₁₃ | >120 s | >240 | <100 | <100 | — |
| | | min | mm | mm | |
| F₁₄ | >120 s | >240 | <100 | <100 | — |
| | | min | mm | mm | |

TABLE 14

| | (Tₑₓₚ) | (T_{1/2}) | (Hₜₒ) | (Hₜ₃₀) | (ΔH) |
|---|---|---|---|---|---|
| F₁₅ | 50 s | >240 | 90 | 65 | 25 |
| | | min | mm | mm | mm |
| F₁₆ | >120 s | >240 | <100 | <100 | — |
| | | min | mm | mm | |
| F₁₇ | >120 s | >240 | <100 | <100 | — |
| | | min | mm | mm | |
| F₁₈ | 80 s | 180 | 80 | 55 | 25 |
| | | min | mm | mm | mm |
| F₁₉ | 60 s | 180 | 75 | 50 | 25 |
| | | min | mm | mm | mm |

TABLE 15

| | (Tₑₓₚ) | (T_{1/2}) | (Hₜₒ) | (Hₜ₃₀) | (ΔH) |
|---|---|---|---|---|---|
| F₂₀ | 7 s | 180 | 138 | 126 | 12 |
| | | min | mm | mm | mm |
| F₂₁ | 4 s | 120 | 144 | 129 | 15 |
| | | min | mm | mm | mm |
| F₂₂ | 5 s | 105 | 144 | 128 | 16 |
| | | min | mm | mm | mm |
| F₂₃ | 6 s | 140 | 135 | 125 | 10 |
| | | min | mm | mm | mm |
| F₂₄ | 4 s | 20 | 168 | 160 | 25 |
| | | min | mm | mm | mm |

4.5) Analyses of the Results

These results show that the compositions E₁ to E₆ according to the invention make it possible to prepare foams having all the qualities required to be used in a process for decontaminating solid surfaces, because they are generated rapidly, in a sufficient volume (100 mm), and are stable (with a half-life of over four hours).

1. A composition (C_{D}) for decontaminating solid surfaces, comprising, per 100% of its mass:
(a)—from 35% to 99.3% by mass of water;
(b)—from 0.5% to 40% by mass of at least one decontamination agent;
(c)—from 0.1% to 10% by mass of at least one gelling agent and/or thickener (AG);
(d)—from 0.1% to 15% by mass of a mixture (M₁) comprising, per 100% of its own mass:
(i)—from 50% to 99% by mass of a composition (C₁) comprising, per 100% of its mass:
(α)—from 65% to 90% by mass of at least one compound of formula (I):
R₁—C(═O)—NH—CH(COOH)—(CH₂)₂—COOH (I)
in acid or partially or totally salified form, wherein the group R₁—C(═O)— represents a linear or branched, saturated or unsaturated acyl radical including from 8 to 18 carbon atoms, and
(β)—from 10% to 35% by mass of at least one compound of formula (II):
R₁—C(═O)—OH (II)
in acid or partially or totally salified form, wherein the group R₁ is as defined for formula (I),
(ii)—from 1% to 50% by mass of a composition (C₂) comprising, per 100% of its mass:
(γ)—from 14% to 80% by mass of a composition (C₃) or of a mixture of compositions (C₃), said composition (C₃) being represented by formula (III):
R₃—O-(G₃)ₚ-H (III)
wherein R₃ represents a linear or branched, saturated or unsaturated aliphatic radical including from 12 to 16 carbon atoms, G₃ represents the residue of a reducing sugar and p represents a decimal number greater than or equal to 1.05 and less than or equal to 5;
(δ)—from 20% to 80% of a composition (C₄) or of a mixture of compositions (C₄), said composition (C₄) being represented by formula (V):
R₄—O-(G₄)_{q}-H (V)
wherein R₄ represents a linear aliphatic radical, chosen from n-butyl (n-C₄H₉—), n-pentyl (n-C₅H₁₁—), n-hexyl (n-C₆H₁₃—) and n-heptyl (n-C₇H₁₅—) radicals, G₄ represents a reducing sugar residue and q represents a decimal number greater than or equal to 1.05 and less than or equal to 5.

2. The composition (C_{D}) as claimed in claim 1, wherein the decontamination agent is chosen from the elements of the group consisting of acidic decontamination agents in acidic or partially or totally salified form; basic decontamination agents; disinfectants.

3. The composition (C_{D}) as claimed in claim 1, wherein the gelling agent and/or thickener is chosen from polysaccharides consisting of monosaccharide derivatives, polysaccharides consisting solely of monosaccharides, cellulose and cellulose derivatives, starches and linear or branched or crosslinked polyelectrolytes.

4. The composition (C_{D}) as claimed in claim 1, wherein said composition (C₃) consists of a mixture of compounds represented by formulae (III₁), (III₂), (III₃), (III₄) and (III₅):
R₃—O-(G₃)₁-H (III₁),
R₃—O-(G₃)₂-H (III₂),
R₃—O-(G₃)₃-H (III₃),
R₃—O-(G₃)₄-H (III₄),
R₃—O-(G₃)₅-H (III₅),
in the respective molar proportions a₁, a₂, a₃, a₄ and as such that:
the sum a₁+a₂+a₃+a₄+a₅ is equal to 1, and
the sum a₁+2a₂+3a₃+4a₄+5a₅ is equal to p.

5. The composition (C_{D}) as claimed in claim 1, wherein said composition (C₄) consists of a mixture of compounds represented by formulae (V₁), (V₂), (V₃), (V₄) and (V₅):
R₄—O-(G₄)₁-H (V₁),
R₄—O-(G₄)₂-H (V₂),
R₄—O-(G₄)₃-H (V₃),
R₄—O-(G₄)₄H (V₄),
R₄—O-(G₄)₅-H (V₅),
in the respective molar proportions a′₁, a′₂, a′₃, a′₄ and a′₅, such that:
the sum a′₁+a′₂+a′₃+a′₄+a′₅ is equal to 1, and
the sum a′₁+2a′₂+3a′₃+4a′₄+5a′₅ is equal to q.

6. The composition (C_{D}) as claimed in claim 1, wherein the composition (C₂) comprises:
from 0% to 3% by mass of at least one alcohol of formula (IV):
R₃—OH (IV)
wherein R₃ is as defined in formula (III), and/or
from 0% to 3% by mass of at least one alcohol of formula (VI):
R₄—OH (VI)
wherein R₄ is as defined for formula (V).

7. The composition (C_{D}) as claimed in claim 1, wherein, in formulae (I) and (II), the group R₁—C(═O)— represents an acyl radical chosen from octanoyl, decanoyl, dodecanoyl, tetradecanoyl, hexadecanoyl, octadecanoyl, 9-octadecanoyl, 9,12-octadecadienoyl and 9,12,15-octadecatrienoyl radicals.

8. The composition (C_{D}) as claimed in claim 1, wherein said composition (C₂) comprises, per 100% of mass:
(γ)—a mass proportion of said composition (C₃) of greater than or equal to 14% and less than 70%, and
(δ)—a mass proportion of said alcohol of formula (IV) of greater than or equal to 0% and less than or equal to 3%,
(ε)—a mass proportion of said composition (C₄) of greater than or equal to 30% and less than or equal to 80%, and
(η)—a mass proportion of said alcohol of formula (VI) of greater than or equal to 0% and less than or equal to 3%.

9. The composition (C_{D}) as claimed in claim 1, wherein, in formulae (III) and (IV), R₃ represents a linear alkyl radical chosen from n-dodecyl (n-C₁₂H₂₅—), n-tetradecyl (n-C₁₄H₂₉—) and n-hexadecyl (n-C₁₆H₃₂—) radicals.

10. The composition (C_{D}) as claimed in claim 1, wherein, in formulae (V) and (VI), R₄ represents a linear alkyl radical chosen from n-hexyl (n-C₆H₁₃—) and n-heptyl (n-C₇H₁₅—) radicals.

11. The composition (C_{D}) as claimed in claim 1, wherein said composition (C₂) comprises a mixture of compositions (C₃) and compositions (C₄), said mixture comprising, per 100% of mass:
(γ₁)—from 13.6% to 44.4% by mass of a composition (C₃) represented by formula (III) wherein R₃ represents the (n-C₁₂H₂₅—) radical,
(γ₂)—from 5% to 16.25% by mass of a composition (C₃) represented by formula (III) wherein R₃ represents the n-tetradecyl (n-C₁₄H₂₉) radical, and
(γ₃)—from 1.4% to 4.55% by mass of a composition (C₃) represented by formula (III) wherein R₃ represents the n-hexadecyl (n-C₁₆H₃₂) radical,
(ε₁)—from 35% to 80% by mass of a composition (C₄) represented by formula (V) in which R₄ represents the n-heptyl radical (n-C₇H₁₅) radical.

12. The composition (C_{D}) as claimed in claim 1, wherein the mass ratio:
Δ=Mass of compound(s) of formula (I)/[Mass of composition (C₃)+Mass of composition (C₄)], is greater than or equal to 20/80 and less than or equal to 65/35.

13. The composition (C_{D}) as claimed in claim 1, wherein the mass ratio:
Δ₁=Mass of composition (C₃)/Mass of composition (C₄) is greater than or equal to 20/80 and less than or equal to 70/30.

14. The composition (C_{D}) as claimed in claim 1, wherein at least one gelling agent and/or thickener (AG) is chosen from xanthan gum (G_{X}), acacia gum exudate (G_{A}), the mixture of xanthan gum (G_{X}) and of acacia gum exudate (G_{A}) in a mass ratio between the xanthan gum (G_{X}) and the acacia gum exudate (G_{A}) which is greater than or equal to 1/3 and less than or equal to 3/1.

15. The composition (C_{D}) as claimed in claim 1, wherein at least decontamination agent is a disinfectant chosen from the elements of the group consisting of chlorinated products, aldehydes and oxidizing agents.

16. The composition (C_{D}) as claimed in claim 1, wherein at least decontamination agent is an acidic agent in acid or partially or totally salified forms, chosen from the elements of the group consisting of hydrochloric acid, nitric acid, sulfuric acid, phosphoric acid, oxalic acid, benzoic acid, sorbic acid, dehydroacetic acid and peracetic acid.

17. The composition (C_{D}) as claimed in claim 1, wherein at least decontamination agent is a basic agent chosen from the elements of the group consisting of sodium hydroxide, potassium hydroxide, sodium carbonate and potassium carbonate.

18. The composition (C_{D}) as claimed in claim 1, comprising, per 100% of mass:
a)—from 35% to 99.3% of water;
b)—from 0.5% to 40% by mass of at least one decontamination agent which is a disinfectant chosen from the group consisting of hydrogen peroxide and sodium hypochlorite;
c)—from 0.1% to 10% by mass of at least one gelling agent and/or thickener (AG) chosen from the elements of the group consisting of xanthan gum (G_{X}), acacia gum exudate (G_{A}), the mixture of xanthan gum (G_{X}) and of acacia gum exudate (G_{A}) in a mass ratio between the xanthan gum (G_{X}) and the acacia gum exudate (G_{A}) which is greater than or equal to 1/3 and less than or equal to 3/1;
d)—from 0.1% to 15% by mass of said mixture (M₁) wherein:
the compound of formula (I) is chosen from monosodium N-cocoyl glutamate, monopotassium N-cocoyl glutamate, disodium N-cocoyl glutamate and dipotassium N-cocoyl glutamate,
the compound of formula (II) is chosen from sodium cocoate and potassium cocoate,
in formula (III), R₃ represents a linear or branched, saturated or unsaturated aliphatic radical including from 12 to 16 carbon atoms, G₃ represents a glucose or xylose residue and p represents a decimal number greater than or equal to 1.05 and less than or equal to 2.5;
in formula (IV), R₃ represents a linear or branched, saturated or unsaturated aliphatic radical including from 12 to 16 carbon atoms,
in formula (V), R₄ represents the n-heptyl (n-C₇H₁₅) radical, G₄ represents a glucose or xylose residue and q represents a decimal number greater than or equal to 1.05 and less than or equal to 2,
in formula (VI), R₄ represents the n-heptyl (n-C₇H₁₅) radical.

19. The composition (C_{D}) as claimed in claim 1, comprising, per 100% of mass:
a)—from 35% to 99.3% of water;
b)—from 0.5% to 40% by mass of at least one decontamination agent which is an acidic agent in acid or partially or totally salified form, chosen from the group consisting of benzoic acid, sorbic acid and dehydroacetic acid;
c)—from 0.1% to 10% by mass of at least one gelling agent and/or thickener (AG) chosen from the elements of the group consisting of xanthan gum (G_{X}), acacia gum exudate (G_{A}), the mixture of xanthan gum (G_{X}) and of acacia gum exudate (G_{A}) in a mass ratio between the xanthan gum (G_{X}) and the acacia gum exudate (G_{A}) which is greater than or equal to 1/3 and less than or equal to 3/1;
d)—from 0.1% to 15% by mass of said mixture (M₁) wherein:
the compound of formula (I) is chosen from monosodium N-cocoyl glutamate, monopotassium N-cocoyl glutamate, disodium N-cocoyl glutamate and dipotassium N-cocoyl glutamate,
the compound of formula (II) is chosen from sodium cocoate and potassium cocoate,
in formula (III), R₃ represents a linear or branched, saturated or unsaturated aliphatic radical including from 12 to 16 carbon atoms, G₃ represents a glucose or xylose residue and p represents a decimal number greater than or equal to 1.05 and less than or equal to 2.5;
in formula (IV), R₃ represents a linear or branched, saturated or unsaturated aliphatic radical including from 12 to 16 carbon atoms,
in formula (V), R₄ represents the n-heptyl (n-C₇H₁₅) radical, G₄ represents a glucose or xylose residue and q represents a decimal number greater than or equal to 1.05 and less than or equal to 2,
in formula (VI), R₄ represents the n-heptyl (n-C₇H₁₅) radical.

20. A process for decontaminating a solid surface, comprising:
at least one step A₁ of preparing a foam obtained by mixing a composition (C_{D}) as defined in claim 1 with a gas or a mixture of gases, chosen from air, nitrogen or carbon dioxide, followed by
at least one step A₂ of bringing the foam prepared in step A₁ into contact with the solid surface.

21. The process for decontaminating a solid surface as defined in claim 20, further comprising a step A₃ of rinsing the solid surface obtained at the end of step A₂.
